(19) United States
(12) Patent Application Publication
(10) Pub. No.: US 2024/0347948 A1
(43) Pub. Date: Oct. 17, 2024

(54) Detachable Connector Receptacles

(71) Applicant: Cadwell Laboratories, Inc., Kennewick, WA (US)

(72) Inventors: Richard A. Villarreal, West Richland, WA (US); David Lee Jepsen, Kennewick, WA (US); David John Yarger, Kennewick, WA (US); Ryan Bowman, Richland, WA (US)

(21) Appl. No.: 18/634,792

(22) Filed: Apr. 12, 2024

Related U.S. Application Data
(63) Continuation-in-part of application No. 18/479,083, filed on Oct. 1, 2023.
Application No. 18/479,083 is a continuation of application No. 17/445,800, filed on Aug. 24, 2021, now Pat. No. 11,777,243.
Application No. 17/445,800 is a continuation of application No. 16/747,748, filed on Jan. 21, 2020, now Pat. No. 11,128,076.
(60) Provisional application No. 63/495,967, filed on Apr. 13, 2023.
(60) Provisional application No. 62/794,884, filed on Jan. 21, 2019.

(51) Int. Cl. H01R 13/11; A61B 5/296; H01R 13/24; H01R 13/52; H01R 107/00

(52) U.S. Cl. CPC H01R 13/111; H01R 13/2485; H01R 13/5202; A61B 5/296; H01R 2107/00

(57) ABSTRACT

A replaceable DIN connector receptacle is provided that may be removably attached to a connector board and coupled with medical equipment in the field, without requiring the connector board or the equipment in which the DIN connector receptacle is housed to be opened. The replaceable DIN connector receptacle allows easy service and maintenance of medical equipment in the field. Relative to convention, DIN connector(s) are removed from the housing of the remaining medical equipment, placed in its own separate housing that is dedicated to just housing the DIN connector(s), and placed in electrical communication with the medical equipment through a dedicated wired or wireless connection.

### CROSS-REFERENCE

The present application relies on U.S. Patent Provisional Application No. 63/495,967, titled “Connector Receptacle”, and filed on Apr. 13, 2023, for priority. In addition, the present application is a continuation-in-part application of U.S. patent application Ser. No. 18/479,083, titled “Method of Connecting Electrical Components” and filed on Oct. 1, 2023, which is a continuation application of U.S. patent application Ser. No. 17/445,800, titled “Connector Receptacle with Improved Mating Retention and Release”, filed on Aug. 24, 2021, and issued as U.S. Pat. No. 11,777,243 on Oct. 3, 2023, which is a continuation application of U.S. patent application Ser. No. 16/747,748, titled “Connector Receptacle”, filed on Jan. 21, 2020, and issued as U.S. Pat. No. 11,128,076 on Sep. 21, 2021, which relies on U.S. Patent Provisional Application No. 62/794,884, of the same title and filed on Jan. 21, 2019, for priority, all of which are herein incorporated by reference in their entirety.

### FIELD

The present specification generally relates to female DIN receptacles, and particularly to a resilient circular female DIN receptacle configured to maintain mating retention and withstand a high number of mating cycles. In addition, the present specification relates to a female DIN receptacle that is easily replaceable without requiring remanufacture or extensive repair of a device in which it is housed.

### BACKGROUND

Electrophysiological methods, such as electroencephalography (EEG), electromyography (EMG), and evoked potentials, measure the functional integrity of certain neural structures (e.g., nerves, spinal cord and parts of the brain). Neurodiagnostics is the use of electrophysiological methods, such as electroencephalography (EEG), electromyography (EMG), and evoked potentials (EP), to diagnose the functional integrity of certain neural structures (e.g., nerves, spinal cord and parts of the brain) to assess disease states and determine potential therapy or treatment. With EMG and EPs, a neurologist typically utilizes electrodes applied to the patient near the nerves being assessed. These electrodes used are in many forms including needles, metal disks, and conductive pads. Each of these electrodes is coupled to a wire lead which, in turn, is connected to an amplifier for amplification of the EMG or EP electrical signals. The wire leads are usually coupled with standard connectors comprising a male plug which fits into a corresponding female receptacle for connecting with the control unit of monitoring equipment, such as, for example, EMG monitoring equipment. The electrical activity pattern captured by various electrodes is analyzed using standard algorithms to localize or spot the portion of brain which is responsible for causing the specific ailment.

During a typical EMG and EP study, the electrodes are connected and disconnected many times, thus requiring a connector that can withstand many mating cycles. After many mating cycles, the connector must maintain reliable electrical connectivity and mating force. An appropriate mating force is necessary since a low mating force may result in the connector being pulled out of the amplifier during the EMG or EP study.

The standard Deutsches Institut für Normung (DIN) connector, for example, a 45322 DIN connector (5 position circular), is a default standard connector used in EMG equipment. Both recording and stimulating electrodes may terminate with male DIN connector plugs. During the course of a patient examination, the connector may be mated/unmated multiple times. The number of mating cycles on a daily basis may exceed 20 depending on the application. Commercially available circular female DIN connector receptacles are typically rated for 1000 mating cycles.

Current methods of securing a DIN connector are not suitable for use with an EMG or EP instrument. These methods include spring fingers and some sort of latching mechanism. Spring fingers have limited mating cycles below what is required for EMG/EP instruments. Latching mechanisms for DIN connectors are not desirable for the following reasons: requires a non-standard DIN plug that would limit the possibility of available electrodes, add cost to the electrode, and require more effort, such as twisting or pressing, to remove the DIN plug from the amplifier.

FIG. 1A illustrates a standard prior art circular male DIN plug, such as a male DIN 45322 plug. FIG. 1B illustrates a prior art female DIN receptacle for the male DIN plug shown in FIG. 1A. In order to enable connection, male DIN plug 102 and female DIN receptacle 104 are mated by fitting the male DIN plug 102 into female DIN receptacle 104. Removing the male DIN plug 102 from the female DIN receptacle 104 results in un-mating or breaking the connection. Beyond 1000 cycles of mating/un-mating, the female DIN receptacle 104 may experience poor electrical contact and retention force of the male DIN connector plug 102.

The retention force of commercially available DIN receptacles relies on the friction between the DIN plug prong and the DIN receptacle pin socket. One type of pin socket is tulip shaped. The poor retention force is due to an elasticity and/or mechanical breakdown of the tulip shaped contacts 106 in the female DIN receptacle 104. The tulip shaped contacts 106 are subject to distortion after repeated mating cycles. Thus, for a female DIN receptacle that is rated for 1000 cycles, the receptacle starts losing its electric contact and retention force after 50 days of use in EMG equipment. Another type of pin socket is a circular contact. Certain variations of the circular contact have superior electrical contact with high mating cycles. However, the retention force of the circular contact is low. Low retention force of the female DIN receptacle is problematic since the electrode may disconnect from the EMG equipment as the electrode is moved about the patient during the examination.

FIG. 1C illustrates a plurality of standard prior art circular female DIN receptacles. FIG. 1C depicts:
A first female DIN receptacle 110, also referred to as a DIN 41524 receptacle, having three contacts 111, each configured to receive a pin;
A second female DIN receptacle 112 having four contacts 113, each configured to receive a pin;
A third female DIN receptacle 114, also referred to as a DIN 45327 receptacle, having five contacts 115, each configured to receive a pin and further configured with a central contact surrounded by four equidistant contacts;
A fourth female DIN receptacle 116, also referred to as a DIN 45322, having five contacts 115, each configured to receive a pin and further configured with a central contact positioned between two right and two left contacts placed in parallel;
A fifth female DIN receptacle 118, also referred to as a DIN 41524 receptacle, having five contacts 115, each configured to receive a pin and further configured in an arc;
A sixth female DIN receptacle 120, also referred to as a DIN 45522 receptacle, having six contacts 121, each configured to receive a pin;
A seventh female DIN receptacle 122, also referred to as a DIN 45329 receptacle, having seven contacts 123;
An eighth female DIN receptacle 124, also referred to as a DIN 45326 receptacle, having eight contacts 125 as shown in FIG. 1C; and
A ninth female DIN receptacle 126 having eight contacts 125 as shown in FIG. 1C.

U.S. Pat. No. 4,284,313, assigned to ITT Corporation, discloses a detachable electrical connector in which “a resilient hood on the receptacle connector member surrounds the forward portion of the plug connector member. Lugs on the plug connector member snap into recesses in the hood to latch the connector members together. The hood is connected to the body of the receptacle member by longitudinally extending ribs. In one embodiment of the invention, the hood is circular and is rotated to ride up over the latching lugs to release the lugs from the recesses, thereby allowing the connector members to be disengaged.”

U.S. Pat. No. 4,842,555, assigned to AMP Incorporated, discloses a surface mounted electrical connector which “has a metal shield (12) having a front plate (28) with a circular opening (30) for receiving a shielded socket (18) of a mating connector (20), to allow it to mate with a plug portion (16) of the surface mounted connector (10). Spring fingers (34) project from the edge (32) of the opening (30) for engaging shielding (19) of the mating connector (20). Between a pair of the fingers (34) there also projects from the edge (32), a latching finger (80) which is shorter than the spring fingers (34), for latching behind a depressable protuberance (90) on the socket (18). When the plug portion (16) is to be mated with the socket (18), a flexible arm (98) in the socket (18), which carries the protuberance (90) is supported by a support bar (88) which is stiffly resilient. During mating, the protuberance (90) displaces the latching finger (80) and latches behind its free end (84). A sleeve (100) on the mating connector (20) can be pulled to withdraw the support bar (98) so that the protuberance can slide under the latching finger (80) to allow the connectors (10 and 20) to be unmated.”

U.S. Pat. No. 5,035,650, assigned to AMP Incorporated, discloses “a dielectric housing (4) of a circular, shielded DIN electrical connector (2) [which] comprises a central block (26) with terminal receiving cavities (18) extending axially therethrough and which cooperates with a hood (30) and a base (28) of the housing (4) to define a circular chamber receiving a circular, stamped and formed metal shield (8). The shield (8) has projecting therefrom a uniplanar latch arm (80) having a head (81) which latches positively against a latching shoulder (88) in a slot in the base. The latch arm (80) is deflected in its own plane by a cam surface in the slot as the latch arm (80) is inserted thereinto and then resiles to allow the head (81) to latch against the shoulder (88).”

U.S. Pat. No. 5,871,371, assigned to The Whitaker Corporation, discloses “an electrical connector for providing connection for a plurality of coaxial cable disposed in a shielded cable. The connector includes a contact housing having a plurality of signal and ground contacts mounted therein. The contact housing has an outer periphery. The signal and ground contacts are disposed about the outer periphery of the contact housing. The signal and ground contacts each have contact sections and connection sections for forming an electrical connection with the signal and ground from a coaxial cable.”

U.S. Pat. No. 6,183,293, assigned to ITT Manufacturing, Inc., discloses an “electrical connector for mounting in an opening (2) of a wall (3), [which] includes a connector element (4) having a bayonet threaded shaft (7) that projects into the opening and a clamp element (5) with a nut part that can be threaded onto the threaded shaft until shoulders on the two elements abut the wall, where the connector includes a latching mechanism (40) that prevents loosening of the clamp member. The latching mechanism includes a holder ring (54) on the connector element, that lies within a latch ring (50) on the clamp element. The latch ring has inwardly-extending projections (42, 44), while the holder ring carries at least one latch member (14). When the clamping member is tightened on the connector element, the distal end (15a) of the latch member readily rides over the projections. However, when the clamping member begins to loosen, the distal end resists such loosening.”

U.S. Pat. No. 9,088,103, assigned to Omnetics Connector Corporation, discloses an “apparatus and system for securing a mated connector pair with a latch. The latch is a unitary elongate body configured to receive the mated connector pair. The latch may include a spring member and an internal passageway configured to receive a miniature circular connector. The latch may include a wire retainer feature to secure the latch to a wire harness.”

The prior art does not address the need for greater retention force between the plug and receptacle of a DIN connector without the use of a latching mechanism. The prior art also does not provide a DIN connector that does not deteriorate by repeated mating cycles of its plug and receptacle, as any connector receptacle will eventually wear out from repeated connector insertions and extractions. Further, the prior art does not allow for in-field replacement of the receptacle of a DIN connector.

Thus, there is a need for a DIN connector receptacle with an improved rating of multiples of 1000 cycles, which is compatible with the standard DIN connector plug to consistently provide a reliable connection over thousands of uses. There is also a need for an improved DIN receptacle that is not worn out by repeated mating and un-mating cycles and that is corrosion resistant. Since receptacles comprising spring leaf type clips tend to flatten out after repeated cycles, resulting in loss of retention force, there is also a need for a receptacle design which would support multiple mating cycles without loss of retention with standard DIN plugs and with no cost increase for the DIN plug. There is also a need for a DIN connector receptacle that is easily replaced in the field without opening the device in which it is housed.

### SUMMARY

The following embodiments and aspects thereof are described and illustrated in conjunction with systems, tools and methods, which are meant to be exemplary and illustrative, not limiting in scope.

In some embodiments, the present specification discloses a medical device configured to receive at least one plug, wherein the medical device comprises: a circuit board comprising a plurality of pins mounted onto a surface of the circuit board; a housing configured to enclose said circuit board such that the plurality of pins are exposed through at least one slot and wherein a circumferential periphery of the at least one slot is defined by said housing; at least one receptacle configured to receive each of the plurality of pins through a first end and further configured to receive the at least one plug through a second end opposing the first end such that, upon insertion into the at least one receptacle, the at least one plug is electrically coupled to the plurality of pins; and at least one connecting member configured to physically attach to the at least one receptacle and physically attach to at least one of the circuit board or the housing.

Optionally, the at least one receptacle is a DIN receptacle.

Optionally, the receptacle is configured to be removable from the at least one slot solely by detaching the at least one connecting member from the circuit board or the housing.

Optionally, at least one connecting member is a screw configured to pass through the at least one receptacle and screw into the circuit board or the housing.

Optionally, the at least one receptacle has a bore positioned in a center of the at least one receptacle and wherein the at least one connecting member is a screw configured to pass through the bore and screw into the circuit board or the housing.

Optionally, the circuit board comprises a second plurality of pins mounted onto the surface of the circuit board, wherein the second plurality of pins is spaced apart from the plurality of pins, wherein the housing is configured to enclose said circuit board such that the second plurality of pins are exposed through a second slot spaced apart from the at least one slot, and wherein a circumferential periphery of the second slot is defined by said housing.

Optionally, the medical device further comprises a second receptacle configured to receive each of the second plurality of pins through a third end and further configured to receive a second plug through a fourth end opposing the third end such that, upon insertion into the second receptacle, the second plug is electrically coupled to the second plurality of pins.

Optionally, the medical device further comprises a second connecting member configured to physically attach to the second receptacle and physically attach to at least one of the circuit board or the housing.

Optionally, the second receptacle is a DIN receptacle.

Optionally, the second receptacle is configured to be removable from the second slot solely by detaching the second connecting member from the circuit board or the housing.

Optionally, the second connecting member is a screw configured to pass through the second receptacle and screw into the circuit board or the housing.

Optionally, the second receptacle has a bore positioned in a center of the second receptacle and wherein the second connecting member is a screw configured to pass through the bore and screw into the circuit board or the housing.

In some other embodiments, the present specification discloses a connector box configured to receive at least one plug, the connector box comprising: a circuit board, wherein the circuit board comprises a first set of pins mounted on a surface of the circuit board; a housing defined by a front portion and a back portion, wherein the front portion comprises at least one slot for receiving the at least one detachable receptacle, wherein the back portion is configured to attach to the front portion, and wherein the circuit board is enclosed in the housing such that the circuit board surface is positioned in the at least one slot and the first set of pins is accessible via the at least one slot; and at least one detachable receptacle having a first end and a second end opposing the first end, wherein the at least one detachable receptacle is configured to receive each of the first set of pins through the first end and configured to receive the at least one plug through the second end such that, upon insertion into the at least one detachable receptacle, the at least one plug is electrically coupled to the first set of pins.

Optionally, the connector box further comprises at least one connecting member configured to physically attach to the at least one detachable receptacle and physically attach to at least one of the circuit board or the housing.

Optionally, the at least one detachable receptacle is configured to be removed from the connector box and replaced with a second detachable receptacle without separating the front portion from the back portion of the connector box.

Optionally, the at least one detachable receptacle comprises an opening positioned centrally to receive the at least one connecting member.

Optionally, each of the first set of pins is a pogo pin and wherein each pogo pin is spring loaded to form an electrical connection with each pin of the at least one plug when mated with the at least one detachable receptacle.

Optionally, the at least one detachable receptacle comprises at least one groove and the connector box comprises at least one ridge, and wherein the at least one groove is configured to slidably engage with the at least one ridge.

In some embodiments, the present specification describes a method of replacing a first connector receptacle attached to a connector box with a second connector receptacle, the method comprising: removing a connecting member from the first connector receptacle to thereby decouple the connecting member from a circuit board positioned in the connector box or a housing of the connector box; removing the first connector receptacle from a slot in the connector box, wherein said removing of the first connector receptacle is achieved without opening the housing or disassembling the connector box; placing the second connector receptacle in said slot such that a back end of the second connector receptacle is positioned over pins extending from a surface of the circuit board; inserting the connecting member or a different connecting member through the second connector receptacle to thereby couple the second connecting member to the circuit board or the housing.

Optionally, the connecting member is a screw and removing the connecting member from the first connector receptacle comprises unscrewing the screw from a central bore passing through the first connector receptacle.

Optionally, the connecting member or different connecting member is a screw and inserting the connecting member or different connecting member comprises screwing the screw through a central bore passing through the second connector receptacle.

In some other embodiments, the present specification discloses a receptacle configured to receive a plug comprising a plurality of pins, wherein the receptacle is coupled with a connector box having a circuit board and wherein the receptacle is replaceable on site without opening the connector box, the receptacle comprising: a top receptacle portion comprising: a first housing portion having a first circular enclosure wall defined by an exterior surface and an interior surface, wherein the interior surface of the first enclosure wall encircles an interior space of the first housing, wherein the first circular enclosure wall comprises a plurality of protrusion openings; a first plug receptacle portion having an exterior surface and positioned within the interior space of the first housing, wherein the interior surface of the first wall circumferentially surrounds the exterior surface of the first plug receptacle portion and is separated from the exterior surface of the first plug receptacle portion by a first circular gap, wherein the first plug receptacle portion comprises a first plurality of hollow connector volumes, and wherein the first plug receptacle portion comprises a first opening portion configured to receive a connecting member to connect the receptacle to the connector box; a plurality of protrusion members positioned in the plurality of protrusion openings; and a retention band circumferentially positioned around first circular enclosure wall, wherein the retention band is configured to physically force the plurality of protrusion members into the first circular gap; a base receptacle portion comprising: a second plurality of hollow connector volumes in line with the first plurality of hollow connector volumes; and a second opening portion in line with the first opening portion and configured to receive a connecting member to connect the receptacle to the connector box; and a plurality of sockets, wherein each socket has a first end configured to be positioned within a one of the first plurality of hollow connector volumes and receive a pin of the plurality of pins of the plug, and a second end opposite the first end and configured to be positioned within a one of the second plurality of hollow connector volumes and receive a pin of the circuit board.

Optionally, the connecting member is a screw configured to screw into the circuit board or a housing of the connector box.

Optionally, the plurality of first protrusion members comprises ball bearings made of steel.

Optionally, the exterior surface of the first circular enclosure wall comprises a first groove and an exterior surface of the base receptacle portion comprises a second groove in line with the first groove, wherein the first groove and second groove are configured to slidably engage with a ridge on the connector box.

Optionally, the receptacle is a female DIN connector receptacle. Optionally, the plug is a male DIN connector plug.

Optionally, the receptacle is configured to connect EMG electrodes with an EMG control system via an amplifier.

Optionally, the retention band is configured to physically force the protrusion members out of the protrusion openings and further into the first circular gap.

Optionally, the receptacle comprises six protrusion members positioned within six protrusion openings, wherein the six protrusion openings are positioned equidistant from each other radially around the first circular enclosure wall. Optionally, the protrusion members are configured to be pressed against an outside surface of the plug by a spring force provided by the retention band.

Optionally, the first housing and the plug receptacle are molded.

Optionally, the retention band is an O-ring having a durometer rating in a range of 50 to 90 Shore.

The present specification also discloses a connector box comprising at least one replaceable connector receptacle, the connector box comprising: a front half comprising at least one slot for receiving the at least one replaceable connector receptacle; a back half configured to attach to the front half; and a circuit board positioned between the front half and the back half, wherein the circuit board comprises a plurality of pins configured to contact and be in electrical communication with the at least one replaceable connector receptacle when the at least on replaceable connector receptacle is received in the at least one slot; wherein the at least one replaceable connector receptacle is configured to be removed from the connector box and replaced with another replaceable connector receptacle without separating the first half of the connector box from the second half of the connector box.

Optionally, the at least one connector receptacle comprises an opening placed centrally to receive a screw for securing the at least one connector receptacle to the connector box.

Optionally, the at least one connector receptacle comprises a plurality of sockets wherein each socket of the plurality of sockets is configured to receive a pin of a connector plug when the connector plug is mated with the at least one connector receptacle.

Optionally, the circuit board comprises a plurality of pogo pins wherein each pogo pin of the plurality of pogo pins is spring loaded to form an electrical connection with each pin of the connector plug when the male connector plug is mated with the at least one connector receptacle.

Optionally, the at least one connector receptacle comprises at least one groove and the connector box comprises at least one ridge, wherein the at least one groove is configured to slidably engage with the at least one ridge.

In some other embodiments, the present specification also discloses a method of replacing a first connector receptacle coupled with a connector box with a second connector receptacle, the method comprising: removing a connector plug connected to a first connector receptacle; removing the first connector receptacle from a slot in the connector box; placing the second connector receptacle in the slot in the connector box; and plugging the connector plug into the second connector receptacle; wherein the first connector receptacle is replaced with the second connector receptacle without disassembling the connector box.

Optionally, removing the first connector receptacle from a slot in the connector box comprises unscrewing a screw attaching the first connector receptacle to the connector box and pulling the first connector receptacle out of the slot.

Optionally, placing the second connector receptacle in the slot in the connector box comprises pushing the second connector receptacle into the slot and screwing a screw through an opening in the second connector receptacle and into the connector box.

In some other embodiments, the present specification discloses a receptacle configured to receive a plug comprising a plurality of pins, wherein the receptacle comprises a first housing having a circular enclosure wall defined by an exterior surface and an interior surface, wherein the interior surface of the enclosure wall encircles an interior space of the first housing, wherein the circular enclosure wall comprises at least one cavity; a plug receiver having an exterior surface and positioned within the interior space of the first housing, wherein the interior surface of the wall circumferentially surrounds the exterior surface of the plug receiver and is separated from the exterior surface of the plug receiver by a circular gap, and wherein the plug receiver comprises a plurality of connectors each configured to receive one of the plurality of pins of the plug; a first protrusion member positioned in the at least one cavity within the circular enclosure wall; and a retention band circumferentially positioned around the interior space and in physical communication with the circular enclosure wall, wherein the retention band is configured to physically force the first protrusion member out of the at least one cavity and into the circular gap.

Optionally, the first protrusion member is a ball bearing.

Optionally, the retention band is positioned within a groove extending circumferentially around the interior surface of the enclosure wall.

Optionally, the receptacle is a female DIN connector receptacle.

Optionally, the plug is a male DIN connector plug.

Optionally, the first protrusion member is a ball bearing and wherein the ball bearing is made of stainless steel.

Optionally, the receptacle is configured to connect EMG electrodes with an EMG control system via an amplifier.

Optionally, the at least one cavity comprises a first cavity and a second cavity and further comprising a second protrusion member positioned in the second cavity, separate and distinct from the first cavity, within the circular enclosure wall.

Optionally, the retention band is configured to physically force the second protrusion member out of the second cavity and further into the circular gap.

Optionally, the first protrusion member and the second protrusion member are positioned 180 degrees from each other on opposite sides of the circular enclosure wall.

Optionally, the first protrusion member is a ball bearing, the second protrusion member is a ball bearing, and both the first protrusion member and second protrusion member are configured to be pressed against an outside surface of the plug by a spring force provided by the retention band.

Optionally, the first housing and the plug receptacle are molded.

Optionally, the retention band is an O-ring having a durometer rating in a range of 50 to 90 Shore.

Optionally, the receptacle further comprises a second protrusion member positioned in a second cavity, separate and distinct from the at least one cavity, within the circular enclosure wall and a third protrusion member positioned in a third cavity, separate and distinct from the second cavity and the at least one cavity, within the circular enclosure wall.

Optionally, the retention band is an O-ring having a durometer rating in a range of 50 to 90 Shore.

Optionally, the retention band is configured to physically force the second protrusion member out of the second cavity and further into the circular gap and to physically force the third protrusion member out of the third cavity and further into the circular gap.

Optionally, the first protrusion member, the second protrusion member, and the third protrusion member are positioned 120 degrees from each other circumferentially around the circular enclosure wall.

In some other embodiments, the present specification discloses a female receptacle configured to connect to a male plug, wherein the female receptacle comprises a first circular housing circumferentially surrounding a plug receptacle, wherein an internal surface of the first circular housing is separated from the plug receptacle by a circular gap and wherein the plug receptacle comprises a plurality of hollow connector spaces each configured to receive a pin of the male plug, the female receptacle further comprising: a first state wherein a first protrusion member is configured to be positioned in a first cavity within the first circular housing and a retention band is configured to be circumferentially positioned around a surface of the first circular housing and physically force the first protrusion member out of the first cavity and further into the circular gap by a first distance; and a second state wherein the first protrusion member is configured to be positioned in the first cavity within the first circular housing and the retention band is configured to be circumferentially positioned around the surface of the first circular housing and physically force the first protrusion member out of the first cavity and further into the circular gap by a second distance, wherein the second distance is less than the first distance and wherein the second state is achieved by inserting the male plug into the female receptacle.

Optionally, the first protrusion member is a ball bearing.

Optionally, the female receptacle further comprises a second cavity, separate and distinct from the first cavity, in the first circular housing and a second protrusion member positioned in the second cavity within the circular enclosure wall.

Optionally, the retention band is configured to physically force the second protrusion member out of the second cavity and further into the circular gap.

In some other embodiments, the present specification discloses a connector receptacle for connecting with a corresponding connector plug coupled with electrodes being used for performing EMG procedure on a patient, the receptacle comprising: a first ball bearing pressing against a first end of a housing of the plug and a second ball bearing pressing against a first end of the housing of the plug when the plug is connected to the receptacle for exerting a retention force against the plug, wherein the first and the second ball bearings are pressed against the first and the second ends respectively by using a spring force.

Optionally, the connector receptacle comprises a high mating cycle circular DIN connector receptacle.

Optionally, said receptacle is compatible with a DIN connector plug.

Optionally, the first and the second ball bearings are made of stainless steel.

Optionally, the spring force is provided by at least one retention band.

Optionally, the retention band comprises a high durometer and low compression set O-ring.

Optionally, the retention band exerts a compression force against the first and the second ball bearings, the compression force being transmitted to the housing of the plug for retaining the plug within the receptacle.

Optionally, the spring force is provided by using a circular spring.

Optionally, the spring force is provided by wounding a circular spring around a center portion of the receptacle for pushing the first and the second ball bearings against the housing of the plug.

Optionally, the spring force is provided by using a coiled spring to push the ball bearings inwards towards the housing of the plug when the plug is connected to the receptacle.

Optionally, the receptacle comprises a molded enclosure encompassing a housing of the receptacle.

The aforementioned and other embodiments of the present specification shall be described in greater depth in the drawings and detailed description provided below.

### BRIEF DESCRIPTION OF THE DRAWINGS

These and other features and advantages of the present specification will be appreciated, as they become better understood by reference to the following detailed description when considered in connection with the accompanying drawings, wherein:

FIG. 1A illustrates a standard prior art male DIN plug;

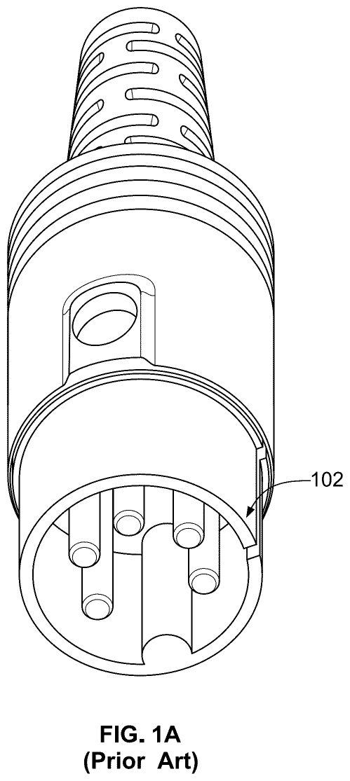

FIG. 1B illustrates a prior art female DIN receptacle for the male DIN plug shown in FIG. 1A;

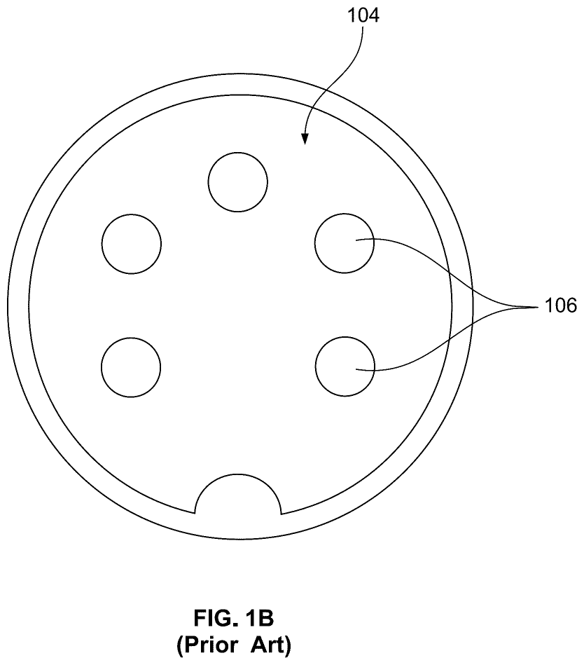

FIG. 1C illustrates a plurality of standard prior art circular female DIN receptacles;

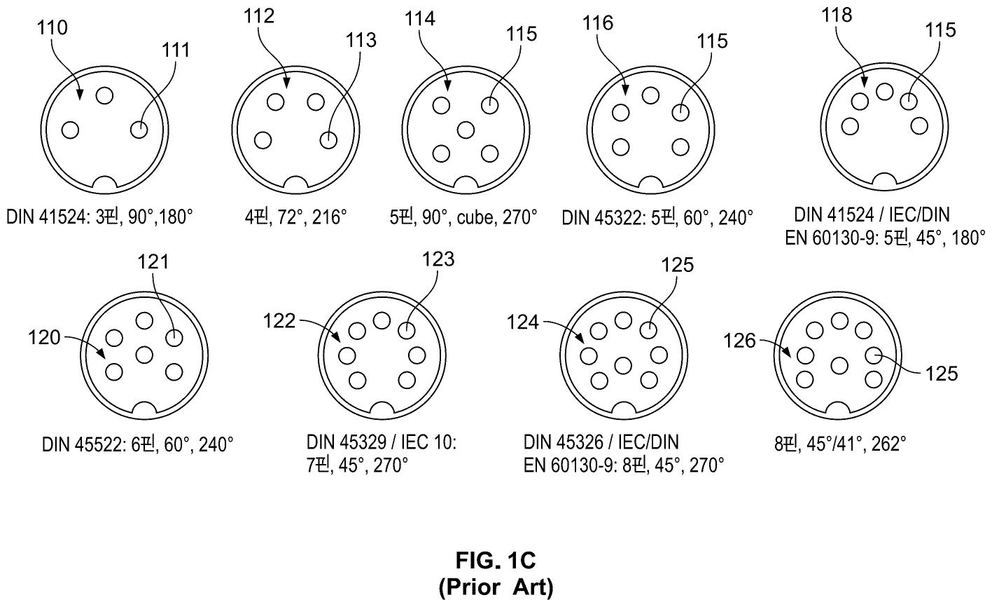

FIG. 2A illustrates a cross sectional diagram of a female DIN receptacle coupled with a corresponding male DIN plug, in accordance with an embodiment of the present specification;

FIG. 2B illustrates a cross sectional diagram of the female DIN receptacle of FIG. 2A without the male DIN plug, in accordance with an embodiment of the present specification;

FIG. 2C is a top view cross sectional diagrammatical representation of the female DIN receptacle of FIG. 2B;

FIG. 3A illustrates equipment comprising an amplifier for performing an EMG procedure on a patient;

FIG. 3B illustrates amplifiers comprising female DIN receptacles, in accordance with an embodiment of the present specification;

FIG. 3C illustrates a close up view of the female DIN receptacles of the amplifier shown in FIG. 3B;

FIG. 3D illustrates a prior art male DIN plug that may be coupled with the male DIN receptacle of FIG. 3C, in accordance with an embodiment of the present specification;

FIG. 3E is a top view cross sectional diagrammatical representation of a female DIN receptacle of FIG. 3C comprising 3 ball bearings, in accordance with an embodiment of the present specification;

FIG. 4A is a perspective view illustration of a connector box comprising at least one replaceable DIN connector receptacle, in accordance with an embodiment of the present specification;

FIG. 4B is an illustration of a replaceable DIN connector, in accordance with an embodiment of the present specification;

FIG. 4C is an exploded view of the components of the replaceable DIN connector shown in FIGS. 4A and 4B;

FIG. 4D illustrates cross-sectional internal views of the replaceable DIN connector shown in FIG. 4B, in accordance with an embodiment of the present specification;

FIG. 4E is an exploded perspective view illustration of the components of the connector box comprising two replaceable DIN connector receptacles shown in FIG. 4A; and

FIG. 4F is a flowchart illustrating a method of replacing a receptacle of a connector box in the field, in accordance with an embodiment of the present specification.

### DETAILED DESCRIPTION

In an embodiment, the present specification provides a high mating cycle circular female DIN connector receptacle. In some embodiments, the circular female DIN receptacle comprises one or more curved or beveled components which press against a housing hood of a corresponding compatible male DIN plug, generating friction for obtaining a greater retention force than that demonstrated by the prior art DIN connectors/receptacles. In various embodiments, the curved or beveled components comprise stainless steel ball bearings. The greater retention force inhibits premature disengagement between a male DIN plug and female DIN receptacle.

In embodiments, the female DIN receptacle of the present specification can be used with any standard male DIN connector and is compatible with standardized male DIN plugs. In an embodiment, the female DIN receptacle of the present specification is used in amplifiers (2 and 12 channels) being used in conjunction with EMG equipment.

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention. In the description and claims of the application, each of the words “comprise” “include” and “have”, and forms thereof, are not necessarily limited to members in a list with which the words may be associated.

It should be noted herein that any feature or component described in association with a specific embodiment may be used and implemented with any other embodiment unless clearly indicated otherwise.

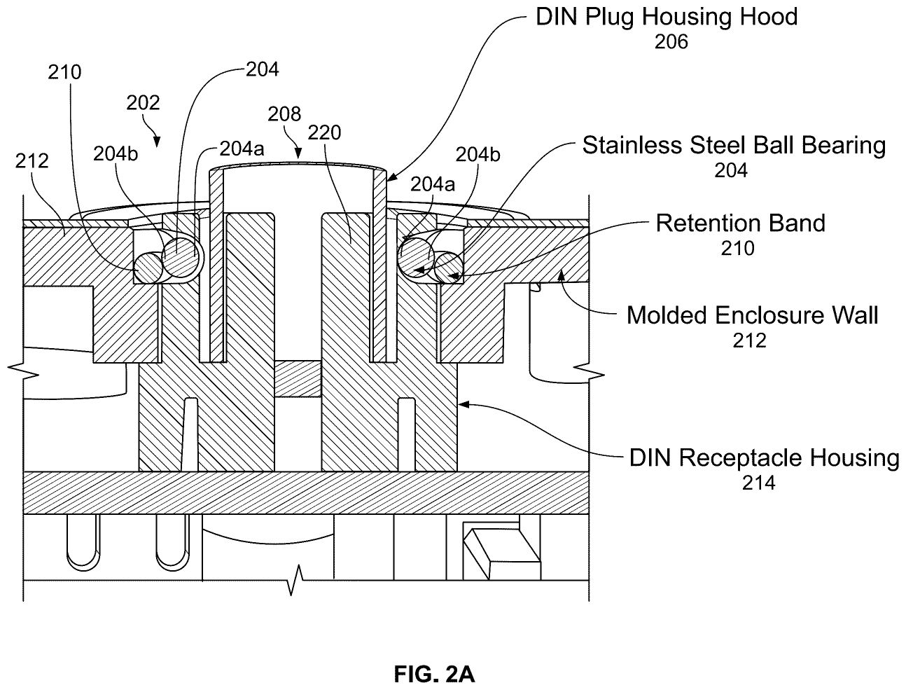

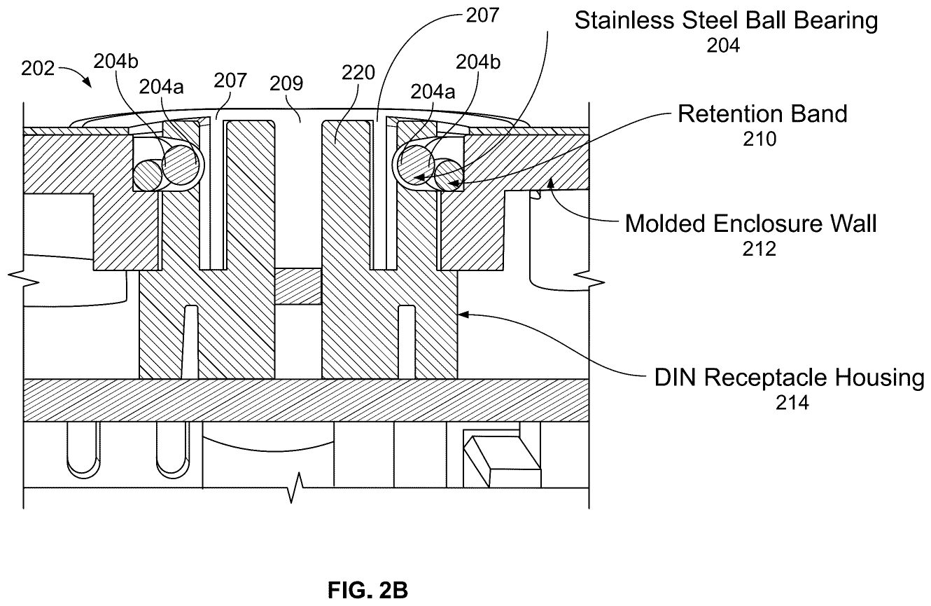

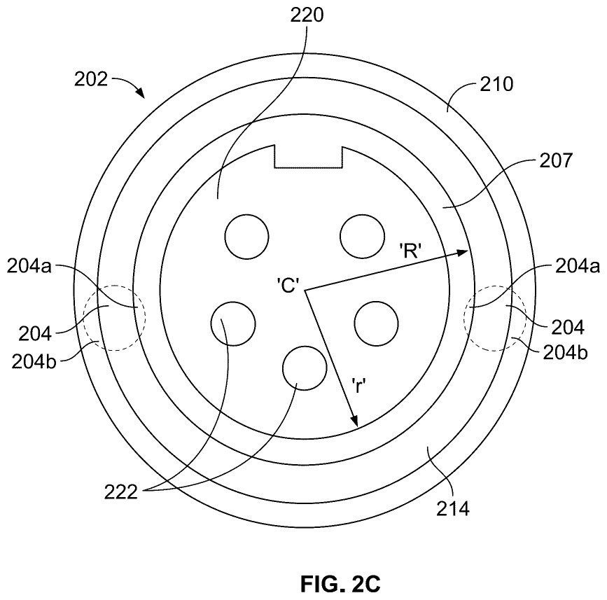

FIG. 2A illustrates a cross sectional diagram of a female DIN connector receptacle 202 coupled with a corresponding male DIN plug 208, in accordance with an embodiment of the present specification. FIG. 2B illustrates a cross sectional diagram of the female DIN connector receptacle 202 of FIG. 2A without the male DIN plug 208, in accordance with an embodiment of the present specification. FIG. 2C is a top view cross sectional diagrammatical representation of the female DIN receptacle of FIG. 2B.

Referring to FIGS. 2A, 2B and 2C, the female DIN receptacle 202 comprises a circular housing 214 circumferentially surrounding a plug receptacle 220. The plug receptacle 220 comprises a plurality of connectors 222 each of which is configured to receive a corresponding pin of the male DIN plug 208. In embodiments, an internal surface of the circular housing 214 is separated from the plug receptacle 220 by a circular gap, well or cavity 207. In some embodiments, the circular housing and the plug receptacle are molded such that they are connected with a gap incorporated therein, as described above.

In some embodiments, the female DIN receptacle 202 further comprises first and second protrusion members 204 positioned, respectively, in first and second gaps, spaces or notches within the circular housing 214. In some embodiments, the first and second protrusion members 204 are positioned 180 degrees from each other (that is, diametrically opposite) around the circular housing 214. In some embodiments, the first protrusion member is a ball bearing. In some embodiments, the first and second protrusion members 204 are ball bearings.

As shown in FIGS. 2A, 2B and 2C, in some embodiments, the first and second protrusion members 204 are ball bearings which press against a housing hood 206 of the corresponding male DIN plug 208, from two opposing sides, when the male DIN plug 208 and the female DIN receptacle 202 are mated. While two ball bearings 204 are depicted in FIG. 2A, one or more ball bearings, such as 2, 3, 4, 5, 6, 7, 8 or more distributed evenly around the circular housing 214 of the female DIN receptacle 202, may be used in various embodiments of the present specification. In an embodiment, the ball bearings 204 are made out of stainless steel. In other embodiments, ball bearings of other suitable materials may also be used such as, but not limited to, chrome steel and ceramic (silicon nitride (Si₃N₄)).

In various embodiments, the ball bearings 204 are pressed against the housing hood 206 by using a spring force. Referring to FIG. 2B, the housing hood (206 of FIG. 2A) of the male DIN plug 208 is inserted into the circular gap 207 for mating/connecting with the female DIN receptacle 202. Also depicted is an opening 209 in the female DIN receptacle 202 for receiving a prong of the male DIN plug 208.

In an embodiment, the spring force is achieved by using a circular retention band or an elastomeric O-ring 210, wherein the retention band 210 presses against each of the ball bearings 204. The circular retention band 210 is circumferentially positioned around an outside surface of the circular housing 214. The retention band 210 is configured to physically force the first and second protrusion members 204 out of their respective first and second gaps and further into the circular gap 207. In some embodiments, when the male DIN plug 208 is not inserted into the female DIN receptacle 202, both the first and second protrusion members 204 are configured to be pressed against an outside surface of the plug receptacle 220 by the spring force provided by the retention band 210. In some embodiments, when the male DIN plug 208 is inserted into the female DIN receptacle 202, both the first and second protrusion members 204 are configured to be pressed against a surface of the male DIN plug 208, such as the housing hood 206, by the spring force provided by the retention band 210.

As can be seen in FIGS. 2A, 2B and 2C, the ball bearings 204 contact the retention band 210 on one end and extend into the gap 207 on the other end to contact the housing hood 206 of the male DIN plug 208 (once inserted) for a secure fit. A first portion 204a of the ball bearings 204 extends into the gap 207 to contact the housing hood 206 of the connector plug 208 when inserted, while a second portion 204b of the ball bearings, opposite the first portion 204a, contacts the compressible retention band 210. When the housing hood 206 of the male DIN plug 208 is inserted into the female DIN receptacle 202, the elastomeric retention band 210 applies pressure to the second portion 204b of the ball bearings, causing the ball bearings to move laterally such that the first portion 204a of the ball bearings contacts and applies a force to the housing hood 206, providing a more secure connection between the male DIN plug 208 and female DIN receptacle 202.

As shown in FIG. 2C, the circular gap 207 has an outer radius ‘R’ and an inner radius ‘r’ such that a width ‘w’ of the circular gap 207 is ‘R-r’. In embodiments, the female DIN receptacle 202 has a first state and a second state. In the first state, the ball bearings 204 are positioned within the circular gap 207 such that X % of the width ‘w’ of the circular gap 207 comprises a ball bearing. In the second state, the ball bearings 204 are positioned within the circular gap 207 such that Y % of the width ‘w’ of the circular gap 207 comprises a ball bearing. In embodiments, Y % is less than X % when the first state corresponds to the male DIN plug 208 not being inserted into the female DIN receptacle 202 whereas the second state corresponds to the housing hood 206 of the male DIN plug 208 being inserted into the female DIN receptacle 202. Thus, in the first state the ball bearings are skewed radially towards a center ‘C’ of the female DIN receptacle 202 (as a result of being pushed by the retention band 210) causing the ball bearings to occupy a higher percentage (X %) of the width ‘w’ of the circular gap 207. However, in the second state the ball bearings are pushed radially away from the center ‘C’, by the inserted hood 206, against the retention band 210 causing the ball bearings to occupy a lesser percentage (Y %) of the width ‘w’ of the circular gap 207.

Stated differently, in the first state, the retention band 210 physically forces the first and second protrusion members 204 out of the respective first and second gaps and further into the circular gap 207 by a first distance. In the second state the retention band 210 physically force the first and second protrusion members 204 out of the respective first and second gaps and further into the circular gap 207 by a second distance. The second distance is less than the first distance when the second state is achieved by the male DIN plug 208 being inserted into the female DIN receptacle 202.

In some embodiments, the retention band 210 has a durometer rating in a range of 50-90 Shore. In an embodiment, the retention band 210 is a high durometer and low compression set O-ring for providing the spring force for pressing the ball bearings 204 against the housing hood 206 of the male DIN plug 208. The retention band 210 is placed in close contact with a ball bearing 204 in order to enable the retention band to press against the ball bearing, thereby creating friction and generating a large retention force between the male DIN plug 208 and the receptacle 202. In some embodiments, approximately 3.68 lbs of force is required to remove a seated male DIN plug 208 from the female DIN receptacle 202, compared to approximately 1.96 lbs of force in the prior art. In some embodiments, a range of 2 lbs or more of force, 2 lbs to 4 lbs of force, or 2 lbs to 5 lbs or force is required to remove a seated male DIN plug 208 from the female DIN receptacle 202, compared to approximately 1.96 lbs of force in the prior art. The retention band 210 is contained within a molded enclosure wall 212 of the female DIN receptacle 202, which also encloses a housing 214 of the female DIN receptacle 202, in order to further enable the retention band 210 to exert a compression force rather than being allowed to expand as the ball bearings 204 are forced out of the gap 207 by the male DIN plug housing hood 206 during disconnection/un-mating.

In another embodiment, a circular spring may be used to generate friction between the ball bearings 204 and the male DIN plug housing hood 206. The circular spring wraps around a center portion or plug receptacle 220 of the female DIN receptacle 202 and presses the ball bearing inward. In other words, a spring force is provided by wounding a circular spring around the center portion or plug receptacle 220 of the female DIN receptacle 202 for pushing the first and the second ball bearings 204 against the housing of the male DIN plug 208. In yet another embodiment, a coiled spring may be used to directly push the ball bearings 204 inward, for generating a retention force between the female DIN receptacle 202 and the male DIN plug 208. Thus, in this embodiment, a spring force is provided by using a coiled spring to push the ball bearings 204 inwards towards the housing of the male DIN plug 208 when the male DIN plug 208 is connected to the female DIN receptacle 202.

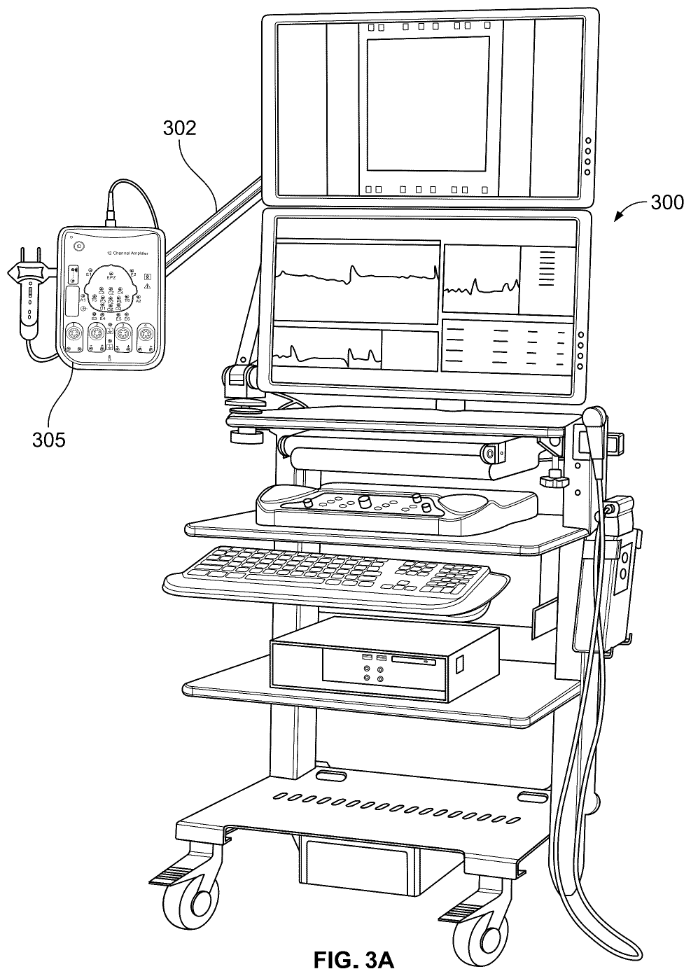

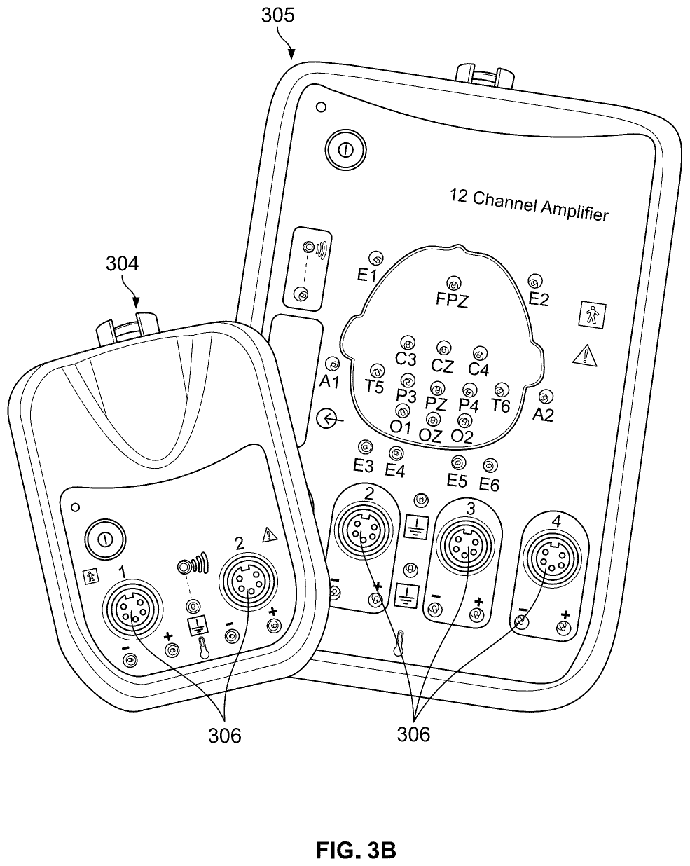

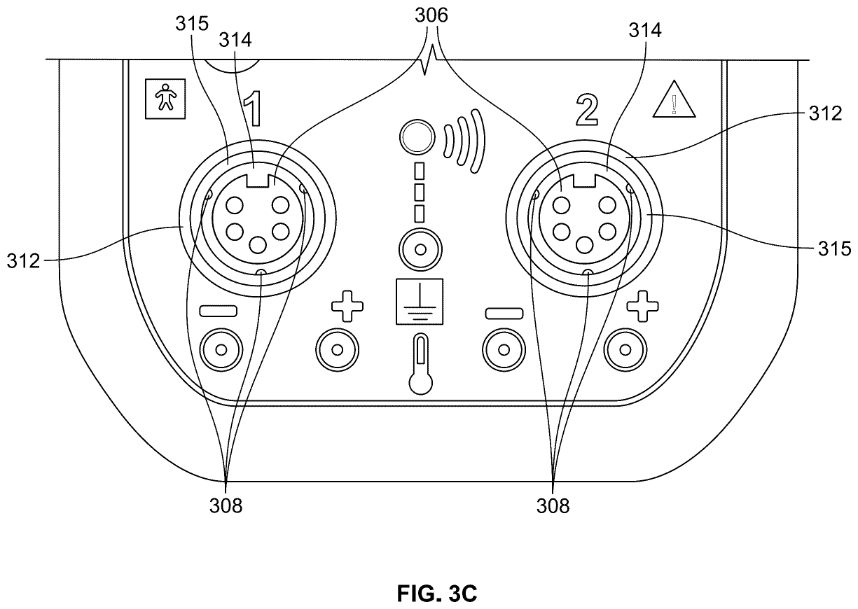

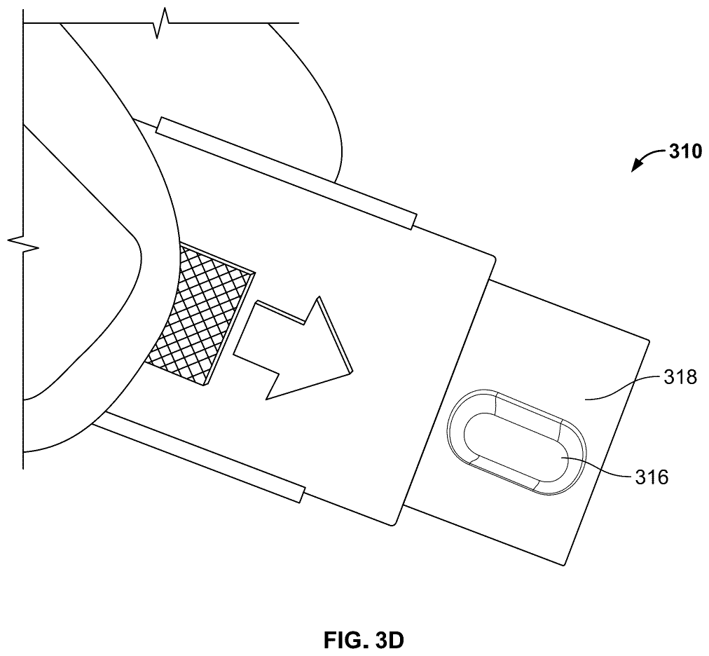

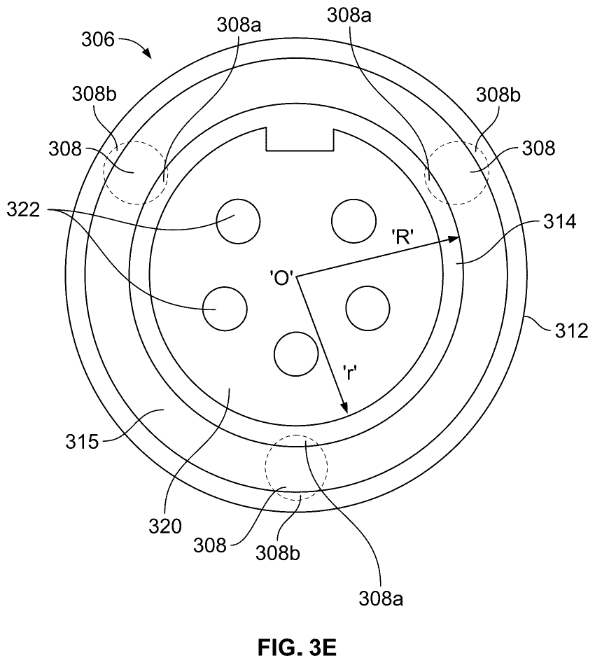

FIG. 3A illustrates an equipment cart 300 comprising an amplifier 305 for performing an EMG procedure on a patient. FIG. 3B illustrates amplifiers 304, 305 comprising female DIN connector receptacles 306, in accordance with an embodiment of the present specification. Referring to FIGS. 3A and 3B, the EMG equipment cart 300 comprises a mobile arm 302 holding the amplifier 305 comprising female DIN connector receptacles 306 for connecting with male DIN connector plugs (not shown in FIGS. 3A, 3B) coupled with electrodes (not shown in FIG. 3A, 3B) being used to monitor a patient's neural activity.

FIG. 3C illustrates a close-up view of the female DIN receptacles 306 of the amplifier 304 shown in FIG. 3B. FIG. 3D illustrates a male DIN plug 310 that may be coupled with any one of the female DIN receptacles 306 of FIG. 3C, in accordance with an embodiment of the present specification. FIG. 3E is a top view cross sectional diagrammatical representation of the female DIN receptacle 306 of FIG. 3C comprising first, second and third protrusion members 308, in accordance with an embodiment of the present specification.

Referring to FIGS. 3C, 3D and 3E, each of the female DIN receptacles 306 comprises a circular housing 315 circumferentially surrounding a plug receptacle 320. The plug receptacle 320 comprises a plurality of hollow connector volumes 322 each of which is configured to receive a corresponding pin of the male DIN plug 310. In embodiments, an internal surface of the circular housing 315 is separated from the plug receptacle 320 by a circular gap, well or cavity 314. In some embodiments, the circular housing and the plug receptacle are molded.

In some embodiments, each of the female DIN receptacles 306 further comprises first, second and third protrusion members 308 positioned, respectively, in first, second and third gaps, spaces or notches within the circular housing 315. In some embodiments, the first, second and third protrusion members 308 are positioned 120 degrees from each other around the circular housing 315. In some embodiments, the first, second and third protrusion members 308 are ball bearings.

In some embodiments, the first, second and third ball bearings 308 press against a housing hood 318 of the corresponding male DIN plug 310 when the male DIN plug 310 and the female DIN receptacle 306 are mated.

In an embodiment, a spring force is achieved by using a circular retention band or an elastomeric O-ring 312, wherein the retention band 312 presses against each of the ball bearings 308. The circular retention band 312 is circumferentially positioned around an outside surface of the circular housing 315. The retention band 312 is configured to physically force the first, second and third protrusion members 308 out of their respective first, second and third gaps and further into the circular gap 314. In some embodiments, when the male DIN plug 310 is not inserted into the female DIN receptacle 306, the first, second and third protrusion members 308 are configured to be pressed against an outside surface of the plug receptacle 320 by the spring force provided by the retention band 312.

As shown in FIG. 3E, the circular gap 314 has an outer radius ‘R’ and an inner radius ‘r’ such that a width ‘w’ of the circular gap 314 is ‘R-r’. In embodiments, each of the female DIN receptacles 306 has a first state and a second state. In the first state, the ball bearings 308 are positioned within the circular gap 314 such that X % of the width ‘w’ of the circular gap 314 comprises a ball bearing. In the second state, the ball bearings 308 are positioned within the circular gap 314 such that Y % of the width ‘w’ of the circular gap 314 comprises a ball bearing. In embodiments, Y % is less than X % when the first state corresponds to the male DIN plug 310 not being inserted into the female DIN receptacle 306 whereas the second state corresponds to the housing hood 318 of the male DIN plug 310 being inserted into the female DIN receptacle 306. Thus, in the first state the ball bearings are skewed radially towards a center ‘O’ of the female DIN receptacle 306 (as a result of being pushed by the retention band 312) causing the ball bearings 308 to occupy a higher percentage (X %) of the width ‘w’ of the circular gap 314. However, in the second state the ball bearings 308 are pushed radially away from the center ‘O’, by the inserted hood 318, against the retention band 312 causing the ball bearings 308 to occupy a lesser percentage (Y %) of the width ‘w’ of the circular gap 314.

Stated differently, in the first state the retention band 312 physically forces the first, second and third protrusion members 308 out of the respective first, second and third gaps and further into the circular gap 314 by a first distance. In the second state the retention band 312 physically forces the first, second and third protrusion members 308 out of the respective first, second and third gaps and further into the circular gap 314 by a second distance. The second distance is less than the first distance when the second state is achieved by the male DIN plug 310 being inserted into the female DIN receptacle 306.

In an embodiment, the ball bearings 308 are positioned approximately 1-3 mm from a top/opening surface of the female DIN receptacle 306. The proximity of the ball bearings 308 to the top/opening surface of the female DIN receptacle 306 allows the user to start the connector engagement process with no physical resistance. When the male DIN plug 310 is not seated in the female DIN receptacle 306, the ball bearings 308 are pushed-in toward the center ‘O’ of the female DIN receptacle 306 (laterally), by the retention band 312, into the circular gap or space 314 configured to receive the housing hood or shell 318 of the male DIN plug 310.

Referring to FIGS. 3D and 3E, and similarly to the embodiment pictured in FIGS. 2A-2C, a first portion 308a (proximal to the center ‘O’ of the female DIN receptacle 306) of the ball bearings 308 extends into the gap 314 to contact the housing hood 318 of the male DIN plug 310 when inserted, while a second portion 308b (distal from the center ‘O’ of the female DIN receptacle 306) of the ball bearings 308, opposite the first portion 308a, contacts the compressible retention band 312. When the housing hood 318 of the male DIN plug 310 is inserted into the female DIN receptacle 306, the elastomeric retention band 312 applies pressure to the second portion 308b of the ball bearings 308, causing the ball bearings 308 to move laterally such that the first portion 308a of the ball bearings 308 contacts and applies a force to the housing hood 318, providing a more secure connection between the male DIN plug 310 and the female DIN receptacle 306.

In embodiments, the retention band 312 is stretched when positioned within the female DIN receptacle 306, so that it is in a tense state. In some embodiments, a dimensional relationship exists between a diameter of the ball bearing 308 and the size of the gap/opening 314 in the female DIN receptacle 306 and/or the housing hood or shell 318 of the male DIN plug 310 (outer circular portion of the male DIN plug 310 that inserts into the female DIN receptacle 306) to ensure that the male DIN plug 310 seats properly and securely within the female DIN receptacle 306. The diameter of the ball bearing 308 is designed such that it is not too large or small. Too large of a diameter will not allow the ball bearing 308 to be pushed aside as the male DIN plug 310 is inserted and too small of a diameter will not allow the ball bearing 308 to cause friction/resistance to hold the male DIN plug 310 in place. In an embodiment, the male DIN plug 310 comprises a dip/recess 316 to accommodate the ball bearing 308 so that the male DIN plug 310 ‘clicks’ into place when seated in the female DIN receptacle 306 when the ball bearings 308 mate with the recess 316.

In various embodiments, the number of ball bearings and/or durometer rating of the retention band may be changed to modulate the force required to remove the male DIN plug from the male DIN receptacle.

Conventionally, DIN connector receptacles coupled with medical equipment, such as, but not limited, to electroencephalogram (EEG) equipment, are a failure point due to contamination or wear and tear during usage. Frequently, a DIN connector receptacle wears out and needs replacement much sooner than the EEG equipment to which the receptacle is connected. The present specification, in an embodiment, provides a replaceable DIN connector receptacle that may be removably attached to a connector board coupled with medical equipment in the field, without requiring any of the connector board, the housing, or the equipment in which the DIN connector receptacle is housed to be opened. The replaceable DIN connector receptacle of the present specification allows for easy service and maintenance of medical equipment in the field.

Accordingly, relative to convention, DIN connector(s) are removed from the housing of the remaining medical equipment, placed in its own separate housing that is dedicated to just housing the DIN connector(s) and no other electrical connectors or processing circuitry, and placed in electrical communication with the medical equipment through a dedicated wired or wireless connection.

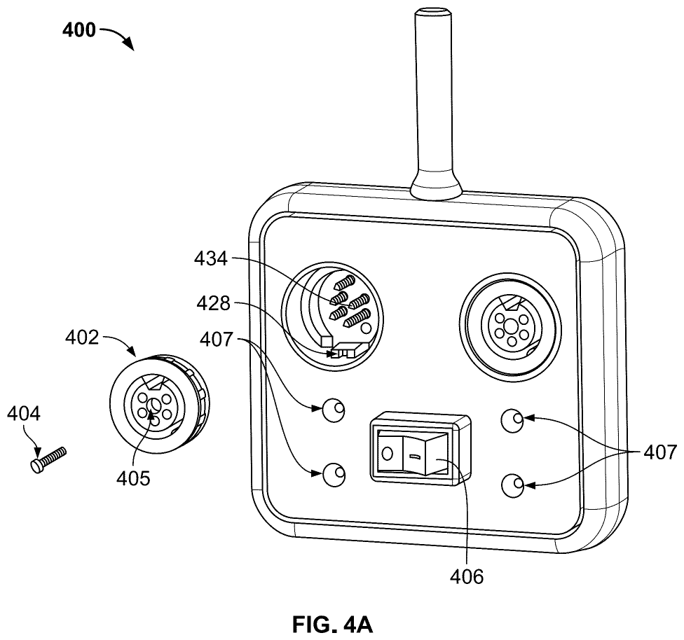

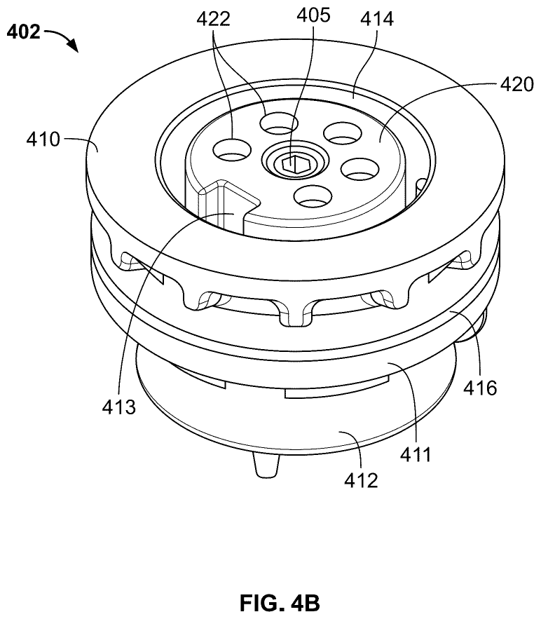

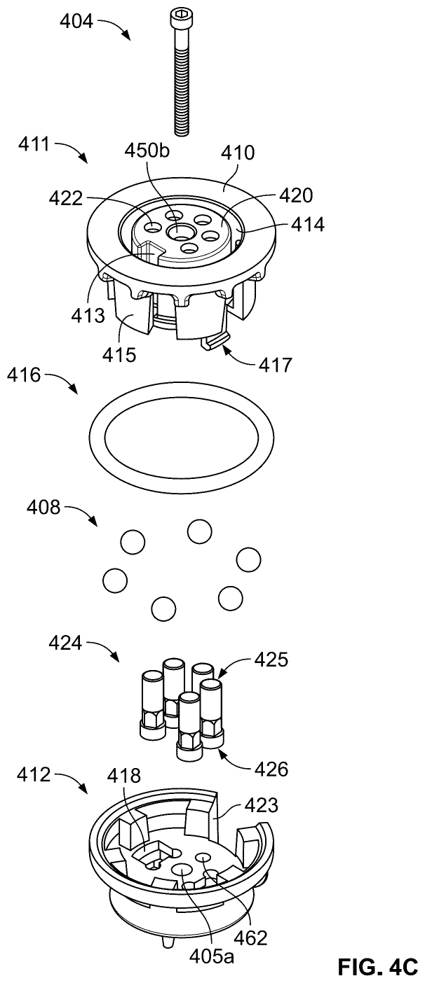

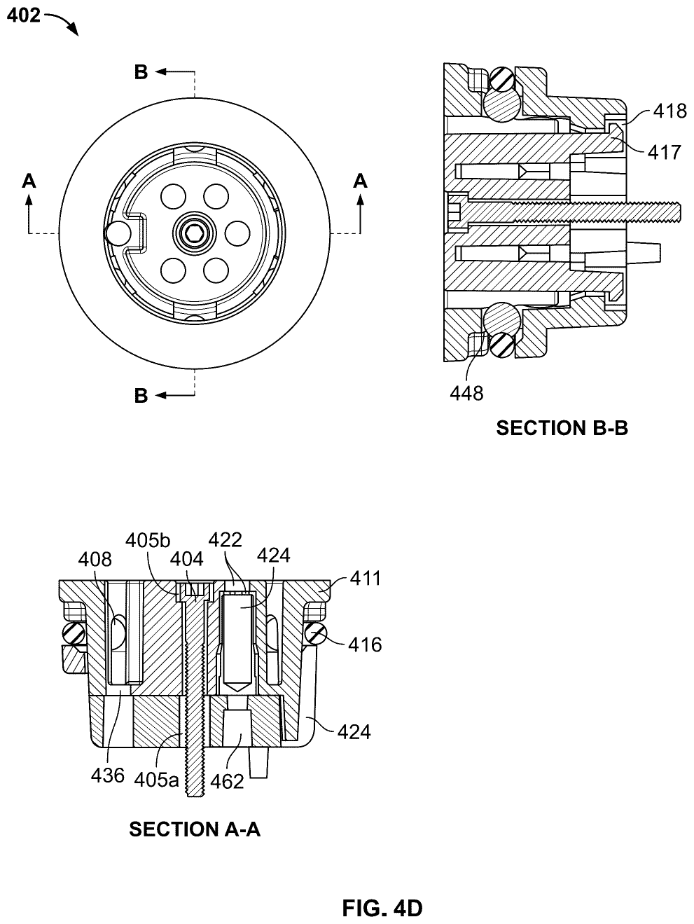

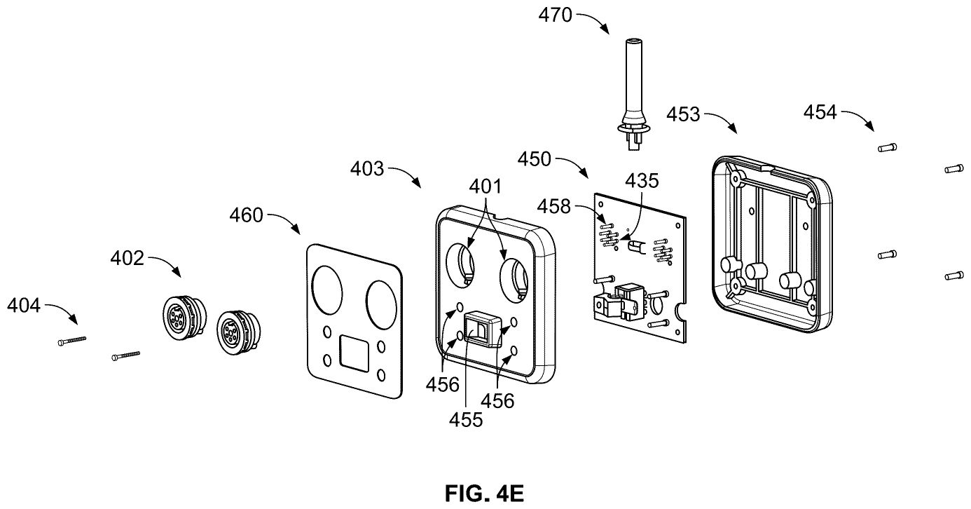

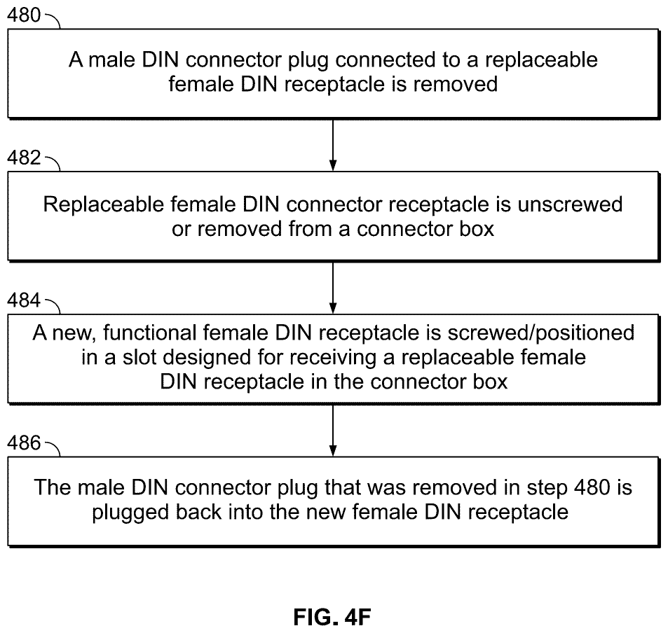

FIG. 4A illustrates a connector box (also referred to as a switch box assembly) 400 comprising at least one replaceable DIN connector receptacle (DIN connector cartridges) 402 while FIG. 4E illustrates various components of the connector box 400, in accordance with an embodiment of the present specification. FIG. 4B is an enlarged illustration of a replaceable DIN connector receptacles, in accordance with an embodiment of the present specification. FIG. 4C is an exploded view of the components of the replaceable DIN connector receptacles shown in FIGS. 4A and 4B while FIG. 4D illustrates cross-sectional internal views of the replaceable DIN connector shown in FIGS. 4A and 4B, in accordance with an embodiment of the present specification. Referring now to FIGS. 4A-4E, simultaneously, the connector box 400 comprises a circuit board 450 enclosed within a housing which is divided into a first portion or front half 403 and a second portion or back half 453, such that the circuit board 450 is sandwiched between the front half 403 and back half 453. In some embodiments, the receptacles 402, the front half 403 and the back half 453 of the housing are made of plastic.

In an embodiment, circuit board 450 is connected to the back half 453 by means of connecting members 454, such as screws. In embodiments, four connecting members 454 are employed. Similarly, the front half 403 is placed over and connected to the back half 453, with circuit board 450 positioned therebetween. The front half 403 comprises slots 401, which, in embodiments are holes that are configured to receive the replaceable female DIN receptacles 402. Each replaceable DIN connector receptacle 402 is connected to the circuit board 450 within the front half 403 via a connecting member 404 which is configured to be inserted through an opening 405 that is included in each of the replaceable DIN receptacles 402 (shown as first opening portion 405a and second opening portion 405b in FIG. 4C) and secured to a receiving captive nut (which includes a hole) 434 in each slot 401. In embodiments, the connecting member 404 is a screw, and in particular a screw that may be used with the DIN connector receptacle. The front half 403 of the housing further comprises a slot 455 (or switch opening) for placement of a circuit board switch 406. In embodiments, the switch 406 is configured to enable the selection of the right or left set of electrode connections. The front half 403 of the housing also comprises a plurality of holes 456, which, in embodiments are touch-proof connection holes for accommodating sockets 407, which, in embodiments, are touch-proof connections configured for connecting the connector box 400 to at least one medical device. In embodiments, each opening 405 is positioned in a center of each receptacle 402 and each receiving captive nut 434 is positioned in a center of each slot 401 such that they can be mated.

The circuit board 450 comprises at least one group of a plurality of spring loaded pogo pins 435, wherein each pogo pin 435 is configured and positioned to make contact with a corresponding socket 424 from below or the bottom end 426 of the socket (FIG. 4C), when the receptacle DIN receptacle 402 is connected to the circuit board 450 within the front half 403 of the housing. In an embodiment, and by way of example only, the circuit board 450 comprises two groups of five spring loaded pogo pins 435. In embodiments, at least one additional pin 458 is positioned within the circuit board 450 above each group of five pogo pins 435 and provides a connection to a cable shield. A label or plate 460 comprising two slots for accommodating each of the replaceable DIN receptacles 402, the switch 406 and the sockets 407 at corresponding positions to the slots formed on the front half 403 of the housing, is placed over the front half 403, after all of the components are connected and the connector box 400 is ready to use, in order to label the DIN receptacles 402, the switch 406 and the sockets 407 in their place within their corresponding slots on the front half 403 of the housing. In an embodiment, the connector box 400 is provided with a cable 470, fitted into the front half 403 and back half 453 of the housing, which is configured to connect with medical devices, such as, but not limited to an EMG or EP monitoring machine.

Each slot 401 is an indent, void, or space configured to receive a DIN receptacle 402 and defined by a plurality of boundaries (including a circumferential boundary) by the front half 403 of the housing and at its base by at least a portion of the circuit board 450. Each slot 401 is configured to receive the DIN connector receptacle 402 such that the DIN connector receptacle 402 it can be easily removed from the slot 401 without opening the front half 403 (and/or the back half 453) of the housing or otherwise disturbing, breaking apart, or separating either or both of the continuity or contiguity of the periphery defining the circumferential boundary of the slot 401 and the base surface of the slot 401, or the circuit board 450.

FIG. 4B illustrates the replaceable DIN connector receptacle 402, FIG. 4C illustrates various components of the replaceable DIN connector receptacle 402, while FIG. 4D illustrates cross-sectional views of the replaceable DIN connector receptacle 402, in accordance with some embodiments of the present specification. Referring now to FIGS. 4A, 4B, 4C and 4D simultaneously, each of the replaceable female DIN receptacles 402 comprises a top receptacle housing portion 411 attached to a base receptacle housing portion 412. The top receptacle housing portion 411 comprises a top receptacle surface portion 410, which, in embodiments, resembles a flat ring. In some embodiments, the top receptacle housing portion 411 comprises at least one first groove 413 positioned within and extending longitudinally along an outer surface of the top receptacle housing portion 411. In embodiments, the base receptacle housing portion 412 comprises at least one second groove 423 positioned within and extending longitudinally along an outer surface of the base receptacle housing portion 412. When the receptacle 402 is assembled, the at least one first groove 413 and at least one second groove 423 align with one another and are configured to slidably engage with a receiving ridge 428 (shown in FIG. 4A) in each slot 401 to properly align each receptacle 402 when inserted into the connector box 400. In embodiments, a latching feature 417 (or latch) of the top receptacle housing portion 411 is configured to be received within a latch cavity 418 of the base receptacle housing portion 412, whereby connecting member 404 (or screw) keeps the two housing portions connected once assembled. The latching feature 417 and the latch cavity 418 keep the two housing portions connected prior to assembly.

The top receptacle housing portion 411 comprises a circular housing portion, surface or enclosure wall 415 defined by an exterior surface and an interior surface, wherein the interior surface encircles an interior space of the top receptacle housing portion 411. A plug receptacle portion 420 (or cable receptacle face) is positioned within the interior space such that the circular housing portion or enclosure wall 415 circumferentially surrounds the plug receptacle portion 420. The circular housing portion 415 includes the top receptacle surface portion 410. In embodiments, the interior surface of the circular housing 415 is separated from an exterior surface of the plug receptacle 420 by a circular gap, well, groove or cavity 414 (cable receptacle groove). The groove 414 is configured to receive an outer portion or housing hood of a male DIN plug (not shown). The plug receptacle portion 420 of the top receptacle housing portion 411 comprises a first plurality of hollow connector volumes or socket cavities 422. Socket 424 floats within hollow connector volume 422, thereby extending the life of the socket 424 since it can be aligned with the pins of the inserted DIN male DIN plug connector. In embodiments, the socket 424 comprises a conductive metal. The base receptacle housing portion 412 comprises a second plurality of hollow connector volumes 462, which serve as holes for pogo pins 435, 458 to pass through to the sockets 424). The first plurality of connector volumes 422 and second plurality of connector volumes 462 are configured to be in-line when the receptacle 402 is assembled. The socket 424 is fitted in a portion of each of the first plurality of hollow connector volumes 422 and a portion of each of the second plurality of hollow connector volumes 462, wherein each socket 424 is configured to receive a corresponding pin of a male DIN plug. Each socket 424 is positioned such that a top end 425 of each socket 424 extends partially into each one of the first plurality of connector volumes 422 and a bottom end 426 of each socket 424 extends partially into each one of the second plurality of hollow connector volumes 462. A first opening portion 405a of opening 405 extends through the plug receptacle 420 and a second opening portion 405b (shown in FIG. 4D) of opening 405, in line with the first opening portion 405a, extends through the base receptacle housing portion 412 for receiving a connecting member 404 for connecting the replaceable female DIN receptacle 402 to the circuit board or the connector box. In embodiments, the connecting member 404 is a screw.

In an embodiment, each of the female DIN receptacles 402 further comprises a plurality of protruding members or steel balls 408 positioned within a plurality of holes or openings 448 (“protrusion openings”), shown in FIG. 4D, that are configured to receive the protruding members or steel balls 408 and located within the circular housing portion 415 proximate the top receptacle surface portion 410. In some embodiments, the circular housing portion 415 comprises six protruding members 408 within six holes or openings 448 configured to receive the protrusions. In some embodiments, the circular housing portion 415 comprises a range of two to ten protruding members and two to ten openings that are configured to receive each of the protrusions. In embodiments, the protrusion openings 448 are positioned equidistant from each other radially around the circular housing portion 415. In some embodiments, the plurality of protruding members 408 are ball bearings. The plurality of protruding members 408 are configured to press against an outer portion or housing hood of a corresponding male DIN plug when the male DIN plug and the female DIN receptacle 402 are mated, securing the male DIN plug in place.

In an embodiment, a spring force is achieved by using a retention band or an elastomeric O-ring 416, wherein the retention band 416 presses against each of the protruding members 408. The retention band 416 is circular, in an embodiment, and circumferentially positioned around an outside surface of the circular housing 415 and under the top receptacle surface portion 410. The retention band 416 is configured to physically force each of the plurality of protruding members 408 partially out of their respectively mated openings 448 configured to receive the protrusions and further into the circular cavity or groove 414. In some embodiments, when a male DIN plug is not inserted into the female DIN receptacle 402, each of the plurality of protruding members 408 is configured to be pressed against an outside surface of the plug receptacle 420 by the spring force provided by the retention band 416. The design and function of the protruding members 408 with respect to the receptacle 402 is additionally described with reference to FIGS. 3D and 3E above.

A pin of a five-pin male DIN connector may be inserted from above and into one of the sockets 424, via a hollow connector volume 422, of the receptacle 402. In embodiments, spring loaded pogo pins 435 are provided on the circuit board 450 to which the receptacle 402 is connected, such that each pogo pin 435 makes contact with a bottom end 426 of each socket 424. In an embodiment, an additional hole 436 is provided in the top receptacle surface portion 410, top receptacle housing portion 411 and the base receptacle portion 412, through which a pogo pin 435 passes through such that it is in contact with the cable shield 470.

FIG. 4F is a flowchart illustrating a method of replacing a receptacle in field with a new receptacle, in accordance with an embodiment of the present specification. In embodiments, a receptacle may be replaced if it is deemed worn and does not provide a reliable connection, faulty, or non-functioning. At step 480, a male DIN connector plug connected to a replaceable female DIN receptacle (such as receptacle 402 shown in FIG. 4E) is removed. At step 482, the replaceable female DIN receptacle is removed or unscrewed from a connector box (such as connector box 400) coupled to the female DIN receptacle. At step 484, a new, functional female DIN receptacle is fastened into a slot (such as slot 401 shown in FIG. 4E) designed for receiving replaceable female DIN receptacle in the connector box. In embodiments, as described with reference to FIGS. 4A-4E, a replaceable DIN receptacle is connected to a circuit board within a housing of the connector box via a screw which is configured to be inserted through an opening in the replaceable DIN receptacle and secured to a receiving captive nut in the slot. At step 486, the male DIN connector plug that was removed in step 480 is plugged back into the new female DIN receptacle.

The above examples are merely illustrative of the many applications of the system of present specification. Although only a few embodiments of the present specification have been described herein, it should be understood that the present specification might be embodied in many other specific forms without departing from the spirit or scope of the specification. Therefore, the present examples and embodiments are to be considered as illustrative and not restrictive, and the specification may be modified within the scope of the appended claims.

We claim:

1. A medical device configured to receive at least one plug, wherein the medical device comprises:
a circuit board comprising a plurality of pins mounted onto a surface of the circuit board;
a housing configured to enclose said circuit board such that the plurality of pins are exposed through at least one slot and wherein a circumferential periphery of the at least one slot is defined by said housing;
at least one receptacle configured to receive each of the plurality of pins through a first end and further configured to receive the at least one plug through a second end opposing the first end such that, upon insertion into the at least one receptacle, the at least one plug is electrically coupled to the plurality of pins; and
at least one connecting member configured to physically attach to the at least one receptacle and physically attach to at least one of the circuit board or the housing.

2. The medical device of claim 1, wherein the at least one receptacle is a DIN receptacle.

3. The medical device of claim 1, wherein the receptacle is configured to be removable from the at least one slot solely by detaching the at least one connecting member from the circuit board or the housing.

4. The medical device of claim 3, wherein the at least one connecting member is a screw configured to pass through the at least one receptacle and screw into the circuit board or the housing.

5. The medical device of claim 1, wherein the at least one receptacle has a bore positioned in a center of the at least one receptacle and wherein the at least one connecting member is a screw configured to pass through the bore and screw into the circuit board or the housing.

6. The medical device of claim 1, wherein the circuit board comprises a second plurality of pins mounted onto the surface of the circuit board, wherein the second plurality of pins is spaced apart from the plurality of pins, wherein the housing is configured to enclose said circuit board such that the second plurality of pins are exposed through a second slot spaced apart from the at least one slot, and wherein a circumferential periphery of the second slot is defined by said housing.

7. The medical device of claim 6, further comprising a second receptacle configured to receive each of the second plurality of pins through a third end and further configured to receive a second plug through a fourth end opposing the third end such that, upon insertion into the second receptacle, the second plug is electrically coupled to the second plurality of pins.

8. The medical device of claim 7, further comprising a second connecting member configured to physically attach to the second receptacle and physically attach to at least one of the circuit board or the housing.

9. The medical device of claim 7, wherein the second receptacle is a DIN receptacle.

10. The medical device of claim 8, wherein the second receptacle is configured to be removable from the second slot solely by detaching the second connecting member from the circuit board or the housing.

11. The medical device of claim 8, wherein the second connecting member is a screw configured to pass through the second receptacle and screw into the circuit board or the housing.

12. The medical device of claim 8, wherein the second receptacle has a bore positioned in a center of the second receptacle and wherein the second connecting member is a screw configured to pass through the bore and screw into the circuit board or the housing.

13. A connector box configured to receive at least one plug, the connector box comprising:
a circuit board, wherein the circuit board comprises a first set of pins mounted on a surface of the circuit board;
a housing defined by a front portion and a back portion, wherein the front portion comprises at least one slot for receiving the at least one detachable receptacle, wherein the back portion is configured to attach to the front portion, and wherein the circuit board is enclosed in the housing such that the circuit board surface is positioned in the at least one slot and the first set of pins is accessible via the at least one slot; and
at least one detachable receptacle having a first end and a second end opposing the first end, wherein the at least one detachable receptacle is configured to receive each of the first set of pins through the first end and configured to receive the at least one plug through the second end such that, upon insertion into the at least one detachable receptacle, the at least one plug is electrically coupled to the first set of pins.

14. The connector box of claim 13, further comprising at least one connecting member configured to physically attach to the at least one detachable receptacle and physically attach to at least one of the circuit board or the housing.

15. The connector box of claim 13, wherein the at least one detachable receptacle is configured to be removed from the connector box and replaced with a second detachable receptacle without separating the front portion from the back portion of the connector box.

16. The connector box of claim 14, wherein the at least one detachable receptacle comprises an opening positioned centrally to receive the at least one connecting member.

17. The connector box of claim 13, wherein each of the first set of pins is a pogo pin and wherein each pogo pin is spring loaded to form an electrical connection with each pin of the at least one plug when mated with the at least one detachable receptacle.

18. The connector box of claim 13, wherein the at least one detachable receptacle comprises at least one groove and the connector box comprises at least one ridge, and wherein the at least one groove is configured to slidably engage with the at least one ridge.

19. A method of replacing a first connector receptacle attached to a connector box with a second connector receptacle, the method comprising:
removing a connecting member from the first connector receptacle to thereby decouple the connecting member from a circuit board positioned in the connector box or a housing of the connector box;
removing the first connector receptacle from a slot in the connector box, wherein said removing of the first connector receptacle is achieved without opening the housing or disassembling the connector box;
placing the second connector receptacle in said slot such that a back end of the second connector receptacle is positioned over pins extending from a surface of the circuit board;
inserting the connecting member or a different connecting member through the second connector receptacle to thereby couple the second connecting member to the circuit board or the housing.

20. The method of claim 19, wherein the connecting member is a screw and removing the connecting member from the first connector receptacle comprises unscrewing the screw from a central bore passing through the first connector receptacle.

21. The method of claim 19, wherein the connecting member or different connecting member is a screw and inserting the connecting member or different connecting member comprises screwing the screw through a central bore passing through the second connector receptacle.
